(19) United States
(12) Patent Application Publication
(10) Pub. No.: US 2025/0057784 A1
(43) Pub. Date: Feb. 20, 2025

(54) TOPICAL COMPOSITION FOR CONTROL OF DEMODEX

(71) Applicant: BRUDER HEALTHCARE COMPANY, LLC, Alpharetta, GA (US)

(72) Inventors: Mark H. Bruder, Alpharetta, GA (US); Rodney L. Dobson, Greer, SC (US); Deborah L. Duffey, Safety Harbor, FL (US); Jeffrey Reynolds, Seminole, FL (US)

(21) Appl. No.: 18/938,860

(22) Filed: Nov. 6, 2024

Related U.S. Application Data
(63) Continuation-in-part of application No. 17/453,239, filed on Nov. 2, 2021.
(60) Provisional application No. 63/108,908, filed on Nov. 3, 2020.

(51) Int. Cl. A61K 31/045; A61K 9/00; A61K 9/127; A61K 35/614; A61K 36/82; A61K 47/24; A61P 33/14

(52) U.S. Cl. CPC A61K 31/045; A61K 9/0014; A61K 9/127; A61K 35/614; A61K 36/82; A61K 47/24; A61P 33/14

(57) ABSTRACT

A topical composition for treating overgrowth of Demodex mites. The topical composition preferably includes one or more active ingredients, optionally encapsulated in lipids. In example embodiments, the topical composition comprises a first active ingredient between either tea tree oil or terpinen-4-ol, and additional active ingredients including sea whip extract, bisabolol, and Camellia sinensis leaf extract.

### CROSS-REFERENCE TO RELATED APPLICATION

This application is a continuation-in-part of U.S. Non-provisional patent application Ser. No. 17/453,239 filed Nov. 2, 2021, which claims the benefit of U.S. Provisional Patent Application Ser. No. 63/108,908 filed Nov. 3, 2020, the entireties of which are hereby incorporated herein by reference for all purposes.

### TECHNICAL FIELD

The present disclosure relates generally to the field of topical preparations, and more particularly to a topical composition for a dermatological preparation comprising a combination of 4-Terpineol, sea whip extract, bisabolol, and Camellia sinensis leaf extract encapsulated in lipids intended for application to a user's skin or hair, such as eyelids and/or eyelashes, to fight the growth of Demodex mites and reduce skin and eye irritation.

### BACKGROUND

Demodex folliculorum and Demodex brevis are two species of Demodex mites typically found on the skin of human and animal subjects. Demodex mites can be found on both symptomatic and asymptomatic individuals and typically pose no serious threat or conditions in low densities. However, infestation of Demodex in excess or high numbers have been implicated in numerous dermatological conditions or demodicosis, especially around the eyes of some individuals, including anterior and posterior blepharitis, dry eye syndrome, meibomian gland dysfunction (MGD), ocular rosacea, blepharoconjunctivitis, and blepharokeratitis, among others.

Conventionally, medicated ointments with active ingredients, such as benzyl benzoate, salicylic acid, selenium sulfide, and sulfur, have been used to combat infestations of Demodex mites and treat demodicosis. However, such ingredients can cause harsh irritation and discomfort if exposed to the eyes of the individual users due to their intrinsic toxicity. Other methods of therapy, such as warm compresses and antibiotic/steroid combinations, may help reduce discomfort for the affected individuals, but such methods do not eradicate the root cause of the demodicosis—the infestation of Demodex mites on the individual's skin.

Currently, a commonly used agent in treating for Demodex is tea tree oil, which comprises the active ingredient terpinen-4-ol, also called terpinen-4-ol (T4O)—a terpene with antimicrobial, antifungal, antiviral, antiseptic, and acaricidal properties. In one study, tea tree oil (TTO) was credited with stimulating Demodex to migrate from the cylindrical dandruff, wherein the Demodex are frequently trapped, to the individuals' skin. See Gao, Y. Y., Di Pascuale, M. A., Li, W., Baradaran-Rafii, A., Elizondo, A., Kuo, C. L., Raju, V. K., & Tseng, S. C. (2005), In Vitro and in Vivo Killing of Ocular Demodex by Tea Tree Oil, The British Journal of Ophthalmology, 89 (11), 1468-1473. The study further found that 10% concentration of TTO was effective in arousing the Demodex to migrate out to the skin for mechanical removal but ineffective in killing the Demodex. 50% concentration of TTO, on the other hand, was much more effective in killing the Demodex but it also generated variable degrees of irritation in some individuals. Most commercially available TTO products contain 1.5% concentration or less of TTO to prevent any burning sensation or discomfort to the users. Therefore, for treatments—such as for Demodex—where user compliance is critical to its overall effectiveness, minimizing discomfort to the patients or users requires significant consideration.

Accordingly, it can be seen that needs exist for improved agents and methods for treatment of Demodex. It is to the provision of treatments meeting these and other needs that the present invention is primarily directed.

### SUMMARY

In example embodiments, the present disclosure provides a topical composition for treatment of Demodex mites. The topical composition includes at least a first active ingredient encapsulated in a nanolipid liposome. In example embodiments, the first active ingredient is terpinen-4-ol, an extract component of tea tree oil. In example embodiments, additional active ingredients are encapsulated in a hydrophilic core of the nanolipid liposome. The additional active ingredients can include one or more of sea whip extract, bisabolol, and Camellia sinensis leaf (green tea) extract. In a particular embodiment, the topical composition includes all four active ingredients, namely terpinen-4-ol (also referred to as 4-Terpineol), sea whip extract, bisabolol, and Camellia sinensis leaf extract.

In various embodiments, the topical composition may include a terpinen-4-ol concentration sufficient to penetrate each of a human hair follicle, Demodex exoskeleton, and Demodex eggs, such as between about 1% and 15% (w/w), about 3% and 10% (w/w), or about 5% (w/w). The topical composition may also have a sea whip extract concentration of about 10% and 13% (w/w), about 1% and 3% (w/w), or about 1% (w/w). The topical composition may also have a bisabolol concentration of about 0.1% and 1% (w/w), about 0.1% and 0.3% (w/w), or about 0.1% (w/w). The topical composition may also have a Camellia sinensis leaf (green tea) extract concentration of about 0.1% and 1% (w/w), about 0.1% and 0.3% (w/w), or about 1% (w/w).

In another aspect, the disclosure relates to a dermatological preparation for example for treating Demodex mites. The dermatological preparation includes at least a first active ingredient encapsulated in a nanolipid liposome. In example embodiments, the first active ingredient is terpinen-4-ol. In example embodiments, the dermatological preparation can include additional active ingredients encapsulated in the lipid bilayer of the nanolipid liposome. The additional active ingredients can include one or more of sea whip extract, bisabolol, and Camellia sinensis leaf (green tea) extract. In example embodiments, the dermatological preparation has an encapsulation rate between about 25% and about 95%. In other embodiments, the encapsulation rate is between about 25% and about 80%. In some embodiments, the hydrophilic active ingredients have an encapsulation rate of about 25% and about 80% and the lipophilic active ingredients have an encapsulation rate of about 25% and about 100%.

In still another aspect, the disclosure relates to a method of treating the overgrowth of Demodex mites on individuals. In example forms, the method includes producing a topical composition comprising at least one active ingredient encapsulated in an excipient. The at least one active ingredient is optionally encapsulated in at least one lipid, for example in nano-lipid particles. The method further includes applying the topical composition to a treatment area on an individual. Optionally, the at least one active ingredient is terpinen-4-ol. Optionally, the method may include producing a topical composition comprising terpinen-4-ol and one or more of sea whip extract, bisabolol, and Camellia sinensis leaf extract.

In other aspects, the disclosure relates to methods of making the compositions described herein.

These and other aspects, features and advantages of the invention will be understood with reference to the detailed description herein, and will be realized by means of the various elements and combinations particularly pointed out in the appended claims. It is to be understood that both the foregoing general description and the following detailed description of example embodiments are explanatory of example embodiments of the invention, and are not restrictive of the invention, as claimed.

### DETAILED DESCRIPTION OF EXAMPLE EMBODIMENTS

The present disclosure may be understood more readily by reference to the following detailed description of example embodiments. It is to be understood that this disclosure is not limited to the specific devices, methods, conditions or parameters described and/or shown herein, and that the terminology used herein is for the purpose of describing particular embodiments by way of example only and is not intended to be limiting of the claimed disclosure. Any and all patents and other publications identified in this specification are incorporated by reference as though fully set forth herein.

Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

The following examples are put forth so as to provide those of ordinary skill in the art with a complete disclosure and description of how to perform the methods and use the devices disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

As used herein, the following terms have the meanings ascribed to them unless specified otherwise. In this disclosure, “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Throughout the disclosure, unless otherwise specified, all concentrations and percentages are weight concentration (% w/w).

### Definitions

Topical compositions, as used herein, refer to compositions that are delivered to the user by application to the skin.

Dermatological preparations, as used herein, refer to preparations that are appropriate for use on the skin and are used to treat skin conditions. There are two types of Demodex mites commonly found on the skin of individuals—Demodex folliculorum and Demodex brevis. While Demodex mites can be found on any part of the human body, they are more prevalent in areas immediately surrounding the eyes, such as the eyelids and eyelashes. For example, Demodex folliculorum are commonly found in eyelash follicles and Demodex brevis are found burrowed deep in the sebaceous and meibomian glands where the Demodex mites feed on sebum, a complex mixture of lipids produced and secreted by the sebaceous glands, as their main food source. Typically, Demodex mites are found in low densities and pose no serious threat or adverse condition to individuals. However, Demodex in high densities have been implicated with causing a variety of dermatological conditions, including anterior and posterior blepharitis, dry eye syndrome, meibomian gland dysfunction (MGD), rosacea, blepharoconjunctivitis, and blepharokeratitis, among others.

The present disclosure relates generally to a composition for topical preparations, such as for example lotions, gels, foams, or liquid, for treating overgrowth of Demodex mites and demodicosis. Generally, the topical composition comprises tea terpinen-4-ol (T4O) as a primary active ingredient and sea whip extract, bisabolol, and Camellia sinensis leaf extract as secondary active ingredients. The T4O is encapsulated in nano-lipid particles. The secondary active ingredients can also be encapsulated in the nano-lipid particles.

According to example embodiments of the present disclosure, the primary active ingredient in the topical composition is T4O, a component extract from tea tree oil known to be an effective agent for treating Demodex mites. The concentration of T4O is directly related to the effectiveness of the topical composition in treating for Demodex mites wherein, generally, a TTO concentration of about 25% or greater is needed for a reasonably effective treatment for Demodex, while a T4O concentration of about 5% could yield similar treatment results. However, high concentrations of T4O may also cause discomfort to the users, such as for example creating burning and/or stinging sensations where applied or variable degrees of irritation of the eyes. As a result, most TTO products contain 1.5% or less of TTO concentration to prevent such discomforts at the cost of diminishing the effectiveness of the treatment at such low concentrations. The topical composition of the present disclosure seeks to provide a balance between an effective acaricide and preventing or minimizing discomfort to its users. In example embodiments of the present disclosure, the topical composition comprises between about 1% and 15% concentration of T4O, between about 3% and 10% concentration of T4O, or more preferably about 5% concentration of T40.

According to example embodiments, the topical composition further comprises one or more of sea whip extract, bisabolol, and Camellia sinensis leaf extract (green tea) as secondary active ingredients. Sea whip extract is a derivative of soft coral (Pseudopterogorgia elisabethae) and provides a soothing and calming effect for irritated eyes and skin. In example embodiments, the topical composition comprises between about 1% and 13% (w/w), about 1% and 3% (w/w), or about 1% (w/w) of sea whip extract. Bisabolol is a plant-derived terpene that provides anti-irritant, anti-inflammatory, and anti-microbial effects. In example embodiments, the topical composition comprises between about 0.1% and 1% (w/w), about 0.1% and 0.3% (w/w), or about 0.1% (w/w) of bisabolol. Camellia sinensis leaf extract provides an analgesic and anti-irritant effect. In example embodiments, the topical composition comprises between 0.1% and 1% (w/w), about 0.1% and 0.3% (w/w), or about 1% (w/w) of Camellia sinensis leaf extract. In example embodiments, the topical composition comprises all of the secondary active ingredients.

Advantageously, because of the anti-inflammatory and analgesic effects of the secondary active ingredients, users can tolerate a higher concentration of T4O and therefore experience faster mite eradication and fewer unpleasant or intolerable side effects than with compositions having lower T4O concentrations without the secondary active ingredients.

According to example embodiments of the present disclosure, the primary and secondary active ingredients are at least partially encapsulated or entrained in lipids, for example nano-lipid particles (also referred to as nanolipid liposomes). In example embodiments, the nano-lipid particles are surfactant-enhanced phospholipid vesicles. Surfactant-enhanced phospholipid vesicles are phospholipid vesicles that employ a surfactant to complete the encapsulation process for lipophilic passenger substances, which enhances the encapsulation efficiency and prevents precipitation of the passenger molecules. The surfactant-enhanced phospholipid vesicles comprise a phospholipid stock solution having phospholipids, ethanol and water, an ethanol diluent, a lipophilic passenger molecule and a load-enhancing surfactant component. In particular embodiments, the phospholipid is a lecithin or lecithin-derived phospholipid (e.g., soy lecithin, egg-based lecithin, phosphatidylcholine, phosphatidylethanol). In some embodiments, the surfactant can be such as polysorbates, ethoxylated fatty alcohols, or ethoxylated fatty acids. In example embodiments, the encapsulation rate of the passenger molecule, in this case T4O, in the topical composition is between about 25% and about 95%. However, generally, encapsulation rates above 50% are preferred. The process of encapsulation is described in U.S. Pat. No. 11,458,092 entitled “Composition with Enhanced Passenger Molecule Loading,” the contents of which are incorporated in their entireties by reference.

In particular embodiments, the encapsulated T4O nanolipidic particles have a diameter of about 160 nm to 170 nm. Advantageously, the size of the particle enables more rapid uptake by the mite through membrane permeation and penetration, both internally and through the exoskeleton. Because T4O is lipophilic, the T4O nanolipidic particles are encapsulated in the lipid bilayer of the phospholipid vesicles. The secondary active ingredients are hydrophilic, and thus reside in both the contiguous aqueous phase outside of the phospholipid vesicles (e.g. the emulsion), as well as inside the hydrophilic core of the phospoholipid vesicles.

The encapsulation of the T4O and secondary active ingredients provides a number of advantages and benefits for the topical composition. First, encapsulation of the active ingredients results in a reduction of eye irritation and T4O-related odor, which some individuals may find offensive or unpleasant. Encapsulation helps to reduce this odor by rendering the T40 unavailable to volatilize out of the particle. Second, encapsulation of T4O in lipids may provide a more effective treatment for combating the overgrowth of Demodex mites wherein Demodex mites which feed on lipids as their main source of food ingest the lipid encapsulated T4O and thereby killing the mites. Third, encapsulation of the active ingredients in nano-lipid particles can provide more rapid tissue/gland penetration and bioavailability, improving the effectiveness in treating Demodex and the permeation and uptake of the secondary active ingredients to soothe irritated glands and tissues more quickly. Because Demodex mites live at the base of the lash, penetration both through the eyelid and the skin of the mite exoskeleton provide effective treatment. Encapsulation of the active ingredients provides this penetration. For treatments reliant on user compliance, including treatments for Demodex, minimizing discomfort (such as eye irritation and unpleasant odors) and improving user relief can be significant factors in the overall effectiveness or results of such treatments. Fourth, encapsulation of the T4O and secondary active ingredients reduces the rate of oxidation of the active ingredients and thereby enhances product stability and shelf-life of the topical composition, as compared to unencapsulated ingredients. Encapsulation of the T4O allows for improved bioavailability and less user toxicity than the same amount of unencapsulated T4O in a carrier such as an oil or other lipid. (Ashfaq R, et. al. Lipid Nanoparticles: An Effective Tool to Improve the Bioavailability of Nutraceuticals. Int J Mol Sci. 2023 Oct. 30; 24(21):15764).

In various embodiments, the topical composition further comprises a seed oil carrier and/or additional bioactive ingredients. In particular embodiments, the seed oil carrier can be shea butter (Butyrospermum parkii). Bioactive ingredients as used herein refer to emollients that soothe skin, prevent dryness, help to retain moisture, or the like. Advantageously, including these ingredients can offset side effects from the T4O. In various embodiments, the bioactive ingredients can include such as squalane, Olea europaea (olive oil), and shea butter. In various embodiments, the topical composition can include about 1% to 3% w/w squalane. In various embodiments, the topical composition can include about 3% to 10% w/w Olea europaea. In various embodiments, the topical composition can include about 3% to 10% w/w shea butter. In particular embodiments, the topical composition can include about 1% to 3% w/w squalane, about 3% to 10% w/w Olea europaea, and about 3% to 10% w/w shea butter.

In a particular embodiment, the topical composition can comprise about 3% to 10% w/w terpinen-4-ol, about 1% to 3% w/w sea whip extract, about 0.1% to 1% w/w bisabolol, about 0.1% and 1% w/w Camellia sinensis leaf extract, about 1% to 3% w/w squalane, about 3% to 10% w/w Olea europaea, and about 3% to 10% w/w shea butter.

The topical composition can further comprise excipients such as surfactants, emulsifiers, stabilizers, preservatives and combinations thereof. In some examples, the topical composition can comprise 50% w/w or greater of the composition, depending upon the viscosity of the desired composition. In general, the composition preferably will have a pH of about 6 to 8 and a viscosity of about 20 k cPs to 90 k cPs. The following ingredients are non-limiting examples of other excipients that can be included in the topical composition in order to promote desirable characteristics such as shelf stability, viscosity, product feel, and the like. The topical composition can comprise one or more of: about 3% to 10% ethanol w/w, about 3% to 10% w/w Polysorbate 80, about 1% to 3% w/w cetearyl alcohol, about 1% to 3% w/w cetyl alcohol, about 1% to 3% w/w cyclopentasiloxane, about 1% to 3% w/w glyceryl stearate, about 1% to 3% w/w PEG-100 stearate, about 0.3% to 1% w/w lecithin, about 0.3% to 1% w/w phenoxyethanol, about 0.3% to 1% w/w di-PPG-3 myristyl ether adipate, about 0.3% to 1% w/w polysorbate 60, about 0.3% to 1% w/w hydrogenated polyisobutene, about 0.3% to 1% w/w propylene glycol, about 0.3% to 1% w/w carbomer, about 0.3% to 1% w/w sodium hydroxide, about 0.3% to 1% w/w PEG-150 Stearate, about 0.3% to 1% w/w Steareth-20, about 0.3% to 1% w/w dimethicone crosspolymer-3, and about 0.3% to 1% w/w ethylhexylglycerin.

In a particular embodiment, the topical composition comprises or consists essentially of about 3% to 10% w/w terpinen-4-ol, about 1% to 3% w/w sea whip extract, about 0.1% to 1% w/w bisabolol, about 0.1% and 1% w/w Camellia sinensis leaf extract, about 1% to 3% w/w squalane, about 3% to 10% w/w Olea europaea, about 3% to 10% w/w shea butter; and about 0.3% to 1% w/w lecithin.

In a particular embodiment, the topical composition comprises or consists essentially of about 3% to 10% w/w encapsulated terpinen-4-ol, about 1% to 3% w/w sea whip extract, about 0.1% to 1% w/w bisabolol, and about 0.1% and 1% w/w Camellia sinensis leaf extract.

In a particular embodiment, the topical composition comprises or consists essentially of about 3% to 10% w/w encapsulated terpinen-4-ol.

In a particular embodiment, the topical composition consists essentially of about 3% to 10% w/w terpinen-4-ol, about 1% to 3% w/w sea whip extract, about 0.1% to 1% w/w bisabolol, about 0.1% and 1% w/w Camellia sinensis leaf extract, about 1% to 3% w/w squalane, about 3% to 10% w/w Olea europaea, about 3% to 10% w/w shea butter, about 60% to 80% water, about 3% to 10% alcohol w/w, about 3% to 10% w/w Polysorbate 80, about 1% to 3% w/w cetearyl alcohol, about 1% to 3% w/w cetyl alcohol, about 1% to 3% w/w cyclopentasiloxane, about 1% to 3% w/w glyceryl stearate, about 1% to 3% w/w PEG-100 stearate, about 0.3% to 1% w/w lecithin, about 0.3% to 1% w/w phenoxyethanol, about 0.3% to 1% w/w di-PPG-3 myristyl ether adipate, about 0.3% to 1% w/w polysorbate 60, about 0.3% to 1% w/w hydrogenated polyisobutene, about 0.3% to 1% w/w propylene glycol, about 0.3% to 1% w/w carbomer, about 0.3% to 1% w/w sodium hydroxide, about 0.3% to 1% w/w PEG-150 Stearate, about 0.3% to 1% w/w Steareth-20, about 0.3% to 1% w/w dimethicone crosspolymer-3, and about 0.3% to 1% w/w ethylhexylglycerin.

While other preservatives, surfactants, emulsifiers, stabilizers, humectants, etc. can be substituted for the above ingredients or the amounts changed, such changes could affect the balance and stability of the composition.

In example embodiments, the topical composition is utilized to produce a topical lotion for applying to eyelids and eyelashes to fight overgrowth of Demodex. In other example embodiments, the topical composition may be utilized in producing other forms of topical preparations or delivery means, such as for example foams, gels, liquids, creams, ointments, oils, or other similar means. Such various preparations and delivery means may be incorporated into various forms of applicators, such as for example pre-soaked pads, brush or foam applicators, roll-on applicators, sprays, pump dispensers, or other similar means. In some example embodiments, the topical composition may include additional active ingredients and/or excipients.

In example methods of use, the topical composition is prepared in the form of a dermatological lotion comprising at least some concentration of T4O and at least some concentration of the secondary active ingredients, wherein the T4O and/or secondary active ingredients are encapsulated in lipids, for example in nano-lipid particles, as described above. The dermatological lotion is dispensed onto a clean fingertip of the user or and applied to the affected skin or treatment area, for example the user's eyelids or another part of the user's body affected by demodicosis. Alternatively, the topical lotion may be dispensed onto an applicator sponge, wipe, cotton ball, cotton bud, or other similar applicators for application to the treatment area. According to example methods of use, the topical lotion is applied to the treatment area once per day or more frequently if the overgrowth of Demodex is severe. In some embodiments, the dermatological lotion may only need to be applied once to provide Demodex eradication sufficient to relieve blepharitis symptoms. In other example methods of use, the topical composition may be applied as a prophylactic to prevent recurrence of Meibomian Gland Dysfunction and/or other Demodex-related issues. In yet another example method of use, the topical composition may be used with compresses, such as eye compresses, to further improve or enhance treatments of various dermatological and/or ocular conditions, such as for example MGD, ocular rosacea, blepharitis, and dry eye syndrome.

In a particular embodiment, the dermatological composition can be a light lotion, such as a light ophthalmic lotion. As used herein, light lotion is easy to rub in and does not require aggressive mechanical shear. Typically, Demodex treatments come in the form of drops or ointments. Some users find drops difficult to apply and they tend to run off of the target area before full penetration can be achieved. Ointments are not attractive to many users because of the lasting, waxy feel, especially near the eye. The light lotion formulation includes oil and water in an emulsion and has a low viscosity. The light lotion penetrates without running off of the target area and does not have a waxy feel.

### EXAMPLES

### Example 1

In a proof-of-concept study to describe the demodicidal activity of 5% terpinen-4-ol (T4O) encapsulated in nano-lipidic particle emulsion lotion on Demodex folliculorum, live Demodex mites were obtained from human volunteers by epilating two eyelashes from the upper and lower eyelid of both eyes. See Milton M Hom; In vitro demodicidal activity using high concentration terpinen-4-ol (T4O) encapsulated in nano-lipidic particle emulsion. Invest. Ophthalmol. Vis. Sci. 2024; 65(7):3622.

The mites were immersed in a test solution: 0.07 grams of 5% terpinen-4-ol (T4O) encapsulated in nano-lipidic particle technology. The test solution was included in a lotion consisting of Water, 4-Terpineol, Alcohol, Butyrospermum Parkii (Shea) Butter, Polysorbate80, Olea Europaea(Olive) Fruit Oil, Cetearyl Alcohol, Squalane, Cetyl Alcohol, Cyclopentasiloxane, Glyceryl Stearate, PEG-100 Stearate, Sea Whip Extract, Camellia Sinensis Leaf Extract, Phenoxyethanol, Ethylhexylglycerin, Lecithin, Di-PPG-3 Myristyl Ether Adipate, Polysorbate 60, Hydrogenated Polyisobutene, Propylene Glycol, Carbomer, Sodium Hydroxide, PEG-150 Stearate, Steareth-20, Dimethicone Crosspolymer-3, and Bisabolol. The test solution was diluted with 1 ml of phosphate buffered saline (1×) pH 7.4 for visibility purposes. Samples were observed under the microscope for up to 137 mins, with time-to-death recorded every 30 minutes. Kill time was defined as the elapsed time between the addition of test solution and all cessation of movement of the body, legs, mouth and pedipalps for a minimum of 5-10 minutes.

The study showed that in all 11 mites, the encapsulated 5% terpinen-4-ol (T4O) solution demonstrated democidal activity as early as 30 minutes with 100% of Demodex mites reaching zero movement or death in an average of 96.6 minutes, even when diluted by 50% in a saline-diluted solution for visibility purposes. The 5% terpinen-4-ol (T4O) encapsulated in nano-lipidic particle lotion effectively killed all mite samples within 137 minutes of exposure.

### Example 2

The example relates generally to a composition for topical preparations, such as for example lotions, gels, foams, or liquid, for treating overgrowth of Demodex mites and demodicosis. Generally, the topical composition comprises tea tree oil (TTO) or terpinen-4-ol (T4O) as a primary active ingredient and cannabidiol (CBD) isolate as a secondary active ingredient. The TTO/T4O and CBD components are optionally encapsulated in nano-lipid particles.

According to example embodiments, the primary active ingredient in the topical composition is T4O, a component extract from TTO known to be an effective agent for treating Demodex mites. The concentration of TTO or T4O is directly related to the effectiveness of the topical composition in treating for Demodex mites wherein, generally, a TTO concentration of about 25% or greater is needed for a reasonably effective treatment for Demodex, while a T40 concentration of about 5% could yield similar treatment results. However, high concentrations of TTO or T4O may also cause discomfort to the users, such as for example creating burning and/or stinging sensations where applied or variable degrees of irritation of the eyes. As a result, most TTO products contain 1.5% or less of TTO concentration to prevent such discomforts at the cost of diminishing the effectiveness of the treatment at such low concentrations. The topical composition of the present example seeks to provide a balance between an effective acaricide and preventing or minimizing discomfort to its users. In example embodiments of the present example, the topical composition comprises between about 1% and 50% concentration of T4O or between about 1.5% and about 75% of TTO concentration, or more preferably about 5% concentration of T4O or between about 10% and about 15% of TTO concentration.

According to example embodiments, the topical composition further comprises cannabidiol (CBD) isolate as the secondary active ingredient. CBD isolate, or CBD oil, provides a soothing and calming effect for irritated eyes. In example embodiments, the topical composition comprises between about 0.01% and about 2.0% concentration of CBD oil, or more preferably about 0.5% concentration of CBD oil.

According to example embodiments, the primary and secondary active ingredients—TTO/T4O and CBD, respectively—are at least partially encapsulated or entrained in lipids, for example nano-lipid particles. In example embodiments, the encapsulation rate of T4O and CBD components in the topical composition is between about 25% and about 95%. However, generally, encapsulation rates above 50% may be preferred.

The encapsulation of the TTO/T4O and CBD agents provides a number of advantages and benefits for the topical composition. First, encapsulation of the active ingredients results in a reduction of eye irritation and TTO-related odor which some individuals may find offensive or unpleasant. Second, encapsulation of T4O in lipids may provide a more effective treatment for combating the overgrowth of Demodex mites wherein Demodex mites which feed on lipids as their main source of food ingest the lipid encapsulated TTO/T4O and thereby killing the mites. Third, T4O and CBD oils encapsulated in nano-lipid particles can provide more rapid tissue/gland penetration and bioavailability, improving the effectiveness in treating Demodex and the permeation and uptake of lipid encapsulated CBD to sooth irritated glands and tissues more quickly. For treatments reliant on user compliance, including treatments for Demodex, minimizing discomforts (such as eye irritation and unpleasant odors) and improving user relief can be significant factors in the overall effectiveness or results of such treatments. Fourth, encapsulation of the T4O and CBD agents reduces the rate of oxidation of the active ingredients and thereby enhances product stability and shelf-life of the topical composition, as compared to unencapsulated ingredients.

In example embodiments, the topical composition is utilized to produce a topical lotion for applying to eyelids and eyelashes to fight overgrowth of Demodex. In other example embodiments, the topical composition may be utilized in producing other forms of topical preparations or delivery means, such as for example foams, gels, liquids, creams, ointments, oils, or other similar means. Such various preparations and delivery means may be incorporated into various forms of applicators, such as for example brush or foam applicators, roll-on applicators, sprays, pump dispensers, or other similar means. In some example embodiments, the topical composition may include additional active ingredients and/or excipients. In still other example embodiments, the topical composition may include only one encapsulated active ingredient.

In example methods of use, the topical composition is prepared in the form of a dermatological lotion comprising at least some concentration of TTO or T4O and at least some concentration CBD oil, wherein the TTO, T4O and/or CBD oil are encapsulated in lipids, for example in nano-lipid particles. The dermatological lotion is dispensed onto a clean fingertip of the user and applied to the affected skin or treatment area, for example the user's eyelids or another part of the user's body affected by demodicosis. Alternatively, the topical lotion may be dispensed onto an applicator sponge, wipe, or other similar applicators for application to the treatment area. According to example methods of use, the topical lotion is applied to the treatment area once per day or more frequently if the overgrowth of Demodex is severe. In other example methods of use, the topical composition may be applied as a prophylactic to prevent recurrence of Meibomian Gland Dysfunction and/or other Demodex related issues. In yet another example method of use, the topical composition may be used with compresses, such as eye compresses, to further improve or enhance treatments of various dermatological and/or ocular conditions, such as for example MGD, blepharitis, and dry eye syndrome.

In some embodiments, the treatment amount is enough lotion to form a thin coating to the affected area, such as about 1/16 mL, about ⅛ mL, about ¼ mL, or about ½ mL. In some embodiments, the dermatological lotion may only need to be applied once to provide Demodex eradication sufficient to relieve blepharitis symptoms.

While the invention has been described with reference to example embodiments, it will be understood by those skilled in the art that a variety of modifications, additions and deletions are within the scope of the invention, as defined by the following claims.

What is claimed is:

1. A topical composition for treatment of Demodex mites, the topical composition comprising between 1% and 50% concentration of terpinen-4-ol and one or more secondary active ingredients, wherein the terpinen-4-ol and one or more secondary active ingredients are encapsulated in a surfactant-enhanced phospholipid vesicle.

2. The topical composition of claim 1, wherein the one or more secondary active ingredients are selected from sea whip extract, Camellia sinensis leaf extract, and bisabolol.

3. The topical composition of claim 1, wherein the topical composition comprises about 3% to 10% w/w of the terpinen-4-ol.

4. The topical composition of claim 1, wherein the topical composition comprises about 1% to 3% w/w of the sea whip extract.

5. The topical composition of claim 1, wherein the topical composition comprises about 0.1% to 1% w/w of the bisabolol.

6. The topical composition of claim 1, wherein the topical composition comprises about 0.1% and 1% w/w of the Camellia sinensis leaf extract.

7. The topical composition of claim 1, wherein the topical composition comprises about 5% concentration of the terpinen-4-ol.

8. The topical composition of claim 1, wherein the topical composition comprises about 3% to 10% w/w of the terpinen-4-ol, about 1% to 3% w/w of the sea whip extract, about 0.1% to 1% w/w of the bisabolol, and 0.1% and 1% w/w of the Camellia sinensis leaf extract.

9. The topical composition of claim 1, wherein the topical composition further comprises one or more of a surfactant, an emulsifier, a humectant, a stabilizer, and a preservative.

10. The topical composition of claim 1, wherein the topical composition is a topical lotion.

11. The topical composition of claim 1, wherein the topical composition comprises about 3% to 10% w/w terpinen-4-ol, about 1% to 3% w/w sea whip extract, about 0.1% to 1% w/w bisabolol, about 0.1% and 1% w/w Camellia sinensis leaf extract, about 1% to 3% w/w squalane, about 3% to 10% w/w Olea europaea, about 3% to 10% w/w shea butter; and about 0.3% to 1% w/w lecithin.

12. A dermatological preparation comprising a first concentration of 4-terpinen-4-ol a second concentration of sea whip extract, a third concentration of bisabolol, and a fourth concentration of Camellia sinensis leaf extract, wherein the 4-terpinen-4-ol, the sea whip extract, the bisabolol, and the Camellia sinensis leaf extract are encapsulated in lipids.

13. The dermatological preparation of claim 12, wherein the first concentration of 4-terpinen-4-01 is between 3% and 10%.

14. The dermatological preparation of claim 12, wherein the second concentration of sea whip extract is between 1% to 3% w/w, the third concentration of bisabolol is between 0.1% to 1% w/w, and a fourth concentration of Camellia sinensis leaf extract is between 0.1% to 1% w/w.

15. The dermatological preparation of claim 12, wherein the dermatological preparation comprises an encapsulation rate between 25% and 95%.

16. A method of treating Demodex mites on a user, the method of treatment comprising:
producing a topical composition comprising 4-terpinen-ol and at least one secondary active ingredient, where both the 4-terpinen-ol and the at least one secondary active ingredient are encapsulated in an excipient comprising at least one lipid, and
applying the topical composition to a treatment area on the user.

17. The method of treating Demodex mites of claim 16, wherein the at least one secondary active ingredient comprises one or more of sea whip extract, Camellia sinensis leaf extract, and bisabolol.

18. The method of treating Demodex mites of claim 16, wherein the topical composition is applied at least once per day.

19. The method of treating Demodex mites of claim 18, wherein the treatment area is an eyelid and base of an eyelash of the user.

20. The method of treating Demodex mites of claim 16, wherein the encapsulation is within a surfactant-enhanced phospholipid vesicle comprising lecithin.
